(19) United States
(12) Patent Application Publication
(10) Pub. No.: US 2009/0270477 A1
(43) Pub. Date: Oct. 29, 2009

(54) POLYMORPHS OF A HYDROISOINDOLINE TACHYKININ RECEPTOR ANTAGONIST

(76) Inventors: Jeffrey T. Kuethe, Somerset, NJ (US); Alex M. Chen, Edison, NJ (US)

Correspondence Address: MERCK AND CO., INC, P O BOX 2000, RAHWAY, NJ, 07065-0907, US

(21) Appl. No.: 12/446,996

(22) PCT Filed: Oct. 29, 2007
(86) PCT No.: PCT/US07/22799

§ 371 Date: Apr. 24, 2009

Related U.S. Application Data
(60) Provisional application No. 60/856,107, filed on Nov. 2, 2006.

(51) Int. Cl. A61K 31/4035; C07D 209/44

(52) U.S. Cl. 514/412; 548/515

(57) ABSTRACT

This application is directed to a novel polymorph of a hydroisoindoline tachykinin receptor antagonist having the following structural formula A.

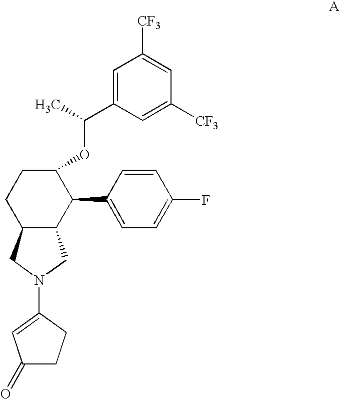

### BACKGROUND OF THE INVENTION

This application is directed to a novel polymorph of a hydroisoindoline tachykinin receptor antagonist having the following structural formula:

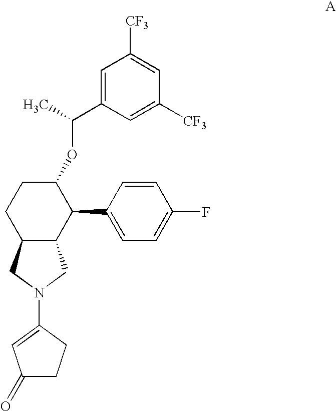

Substance P is a naturally occurring undecapeptide belonging to the tachykinin family of peptides, the latter being so-named because of their prompt contractile action on extravascular smooth muscle tissue. The tachykinins are distinguished by a conserved carboxyl-terminal sequence. In addition to substance P, the known mammalian tachykinins include neurokinin A and neurokinin B. The current nomenclature designates the receptors for substance P, neurokinin A, and neurokinin B as neurokinin-1 (NK-1), neurokinin-2 (NK-2), and neurokinin-3 (NK-3), respectively.

Tachykinin, and in particular substance P, antagonists are useful in the treatment of clinical conditions which are characterized by the presence of an excess of tachykinin, in particular substance P, activity, including disorders of the central nervous system, nociception and pain, gastrointestinal disorders, disorders of bladder function and respiratory diseases.

Structural formula A and methods of making same have previously been described in WO2005/073191, published Aug. 11, 2006 and in US2006/073191, filed Jul. 7, 2006, both of which are incorporated by reference.

### SUMMARY OF THE INVENTION

This application is directed to a novel polymorph of a hydroisoindoline tachykinin receptor antagonist having the following structural formula A:

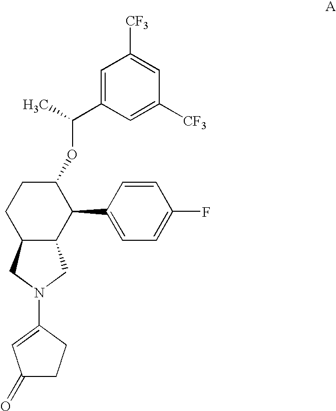

### DETAILED DESCRIPTION OF THE INVENTION

In one aspect the invention is directed to the crystalline anhydrous Form II of the compound structural formula A

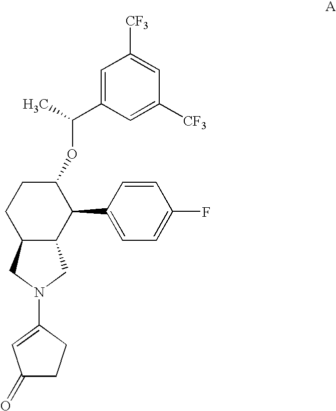

In another aspect, the invention is directed to the crystalline anhydrous Form II of the compound structural formula A

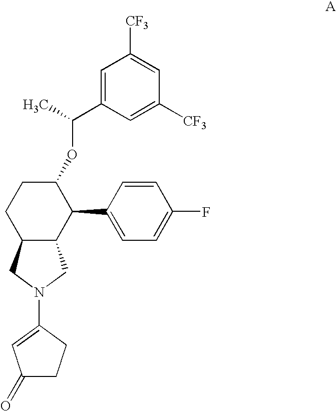

characterized by diffraction peaks obtained from the X-ray powder diffraction pattern corresponding to d-spacings of 7.7, 4.9 and 3.9 angstroms.

Within this aspect, is the genus wherein the crystalline anhydrous Form II of the compound structural formula A

further characterized by diffraction peaks obtained from the X-ray powder diffraction pattern corresponding to d-spacings of 5.3, 4.6 and 3.9 angstroms.

Within this aspect is the genus wherein the crystalline anhydrous Form II of the compound structural formula A

further characterized by diffraction peaks obtained from the X-ray powder diffraction pattern corresponding to d-spacings of 4.2, 3.8 and 2.8 angstroms.

In another aspect, the invention is directed to the crystalline anhydrous Form II of the compound structural formula A

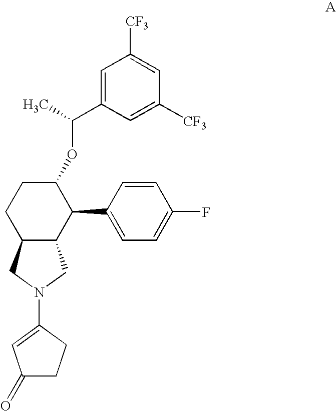

characterized by the X-ray powder diffraction pattern of FIG. 6.

In another aspect, the invention is directed to the crystalline anhydrous Form II of the compound structural formula A

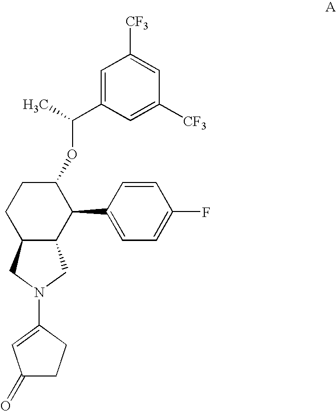

characterized by a solid-state fluorine-19 MAS nuclear magnetic resonance spectrum showing signal at −60.4, −63.4, and −115.3 ppm,

In another aspect the invention is directed to the crystalline anhydrous Form II of the compound structural formula A

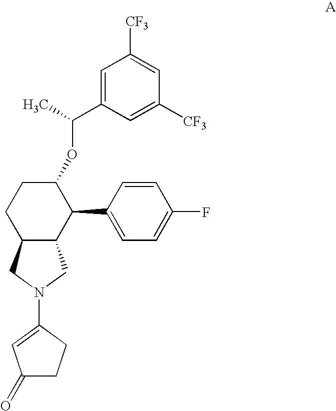

characterized by the solid-state fluorine-19 MAS nuclear magnetic resonance spectrum of FIG. 8.

In another aspect, the invention is directed to the crystalline anhydrous Form II of the compound structural formula A

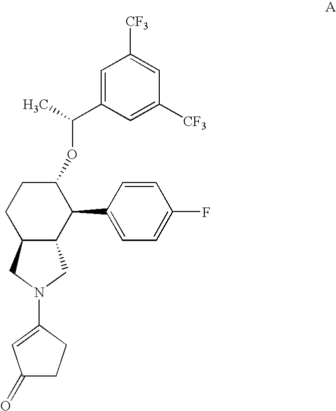

characterized by a melting onset at 218° C.

Within this aspect, the invention is further characterized by a peak temperature of 220.3° C.

Within this aspect the invention is further characterized by an enthalpy change of 71.7 J/g.

Freebase of Compound of Structural Formula A can exist in two anhydrous crystalline forms, Form I and Form II. Form I and Form II are enantiotropic with Form I thermodynamically more stable at temperatures below 72° C. and Form II thermodynamically more stable at temperatures above 72° C.

### BRIEF DESCRIPTION OF THE FIGURES

FIG. 1 is a characteristic X-ray diffraction pattern of the crystalline anhydrous freebase Form I of Compound A.

FIG. 2 is a carbon-13 cross-polarization magic-angle spinning (CPMAS) nuclear magnetic resonance (NMR) spectrum of the crystalline anhydrous freebase Form I of compound A.

FIG. 3 is a fluorine-19 magic-angle spinning (MAS) nuclear magnetic resonance (NMR) spectrum of the crystalline anhydrous freebase Form I of compound A.

FIG. 4 is a typical DSC curve of the crystalline anhydrous freebase Form I of Compound A.

FIG. 5 is a typical thermogravimetric (TG) curve of the crystalline anhydrous freebase Form I of Compound A.

FIG. 6 is a characteristic X-ray diffraction pattern of the crystalline anhydrous freebase Form II of Compound A.

FIG. 7 is a carbon-13 cross-polarization magic-angle spinning (CPMAS) nuclear magnetic resonance (NMR) spectrum of the crystalline anhydrous freebase Form II of compound A.

FIG. 8 is a fluorine-19 magic-angle spinning (MAS) nuclear magnetic resonance (NMR) spectrum of the crystalline anhydrous freebase Form II of compound A.

FIG. 9 is a typical DSC curve of the crystalline anhydrous freebase Form II of Compound I.

FIG. 10 is a typical thermogravimetric (TG) curve of the crystalline anhydrous freebase Form II of Compound A.

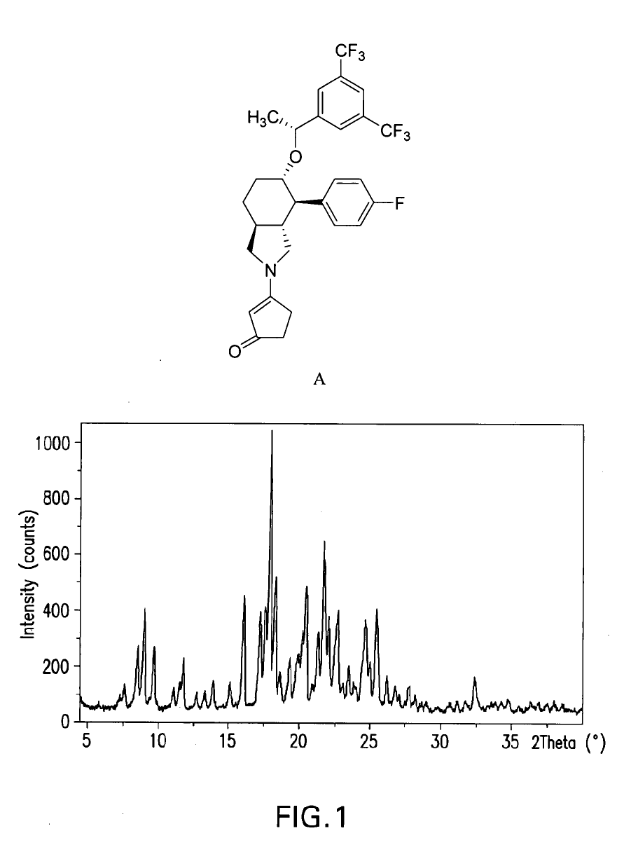

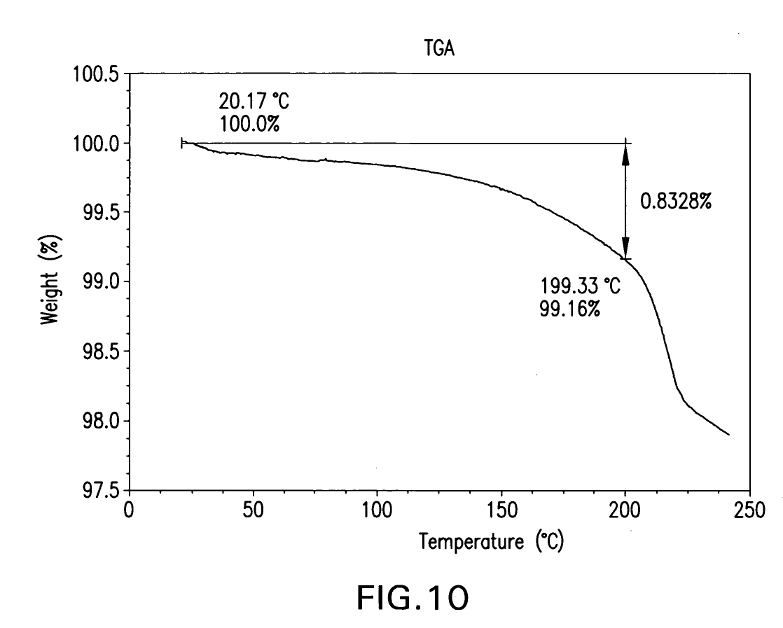

Table: Major peaks for Form I from FIG. 1 are as shown below (wavelength Cu Kalpha).

| 2 theta (degree) | d-spacing (angstroms) |
|---|---|
| 8.5 | 10.4 |
| 16.1 | 5.5 |
| 21.7 | 4.1 |
| 9.0 | 9.9 |
| 9.7 | 9.2 |
| 17.9 | 5.0 |
| 22.7 | 3.9 |
| 24.6 | 3.6 |
| 25.4 | 3.5 |

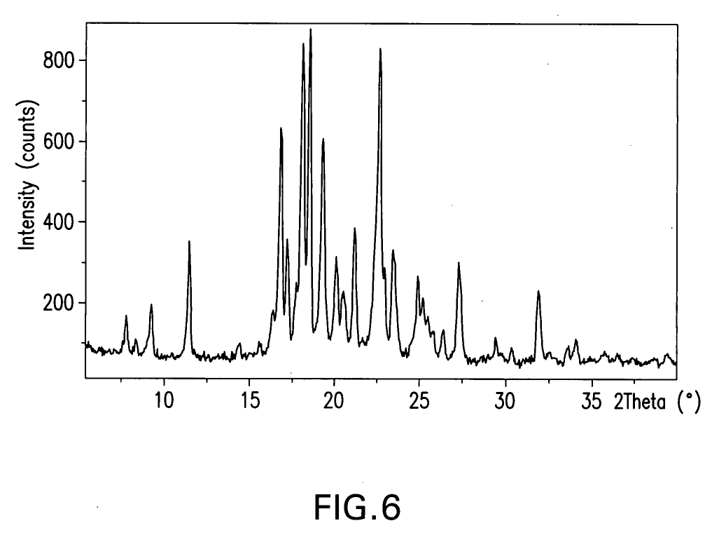

Major peaks for Form II from FIG. 6 are as shown below (wavelength Cu Kalpha):

| 2 theta (degree) | d-spacing (angstroms) |
|---|---|
| 11.4 | 7.7 |
| 18.1 | 4.9 |
| 18.5 | 4.8 |
| 16.8 | 5.3 |
| 19.3 | 4.6 |
| 22.6 | 3.9 |
| 21.2 | 4.2 |
| 23.4 | 3.8 |
| 31.9 | 2.8 |

### DETAILED DESCRIPTION OF THE INVENTION

X-ray powder diffraction studies are widely used to characterize molecular structures, crystallinity, and polymorphism. The X-ray powder diffraction patterns of the crystalline anhydrous Freebase of the present invention were generated on a Philips Analytical X'Pert PRO X-ray Diffraction System with PW3040/60 console. A PW3373/00 ceramic Cu LEF X-ray tube K-Alpha radiation was used as the source.

FIG. 1 shows the X-ray diffraction pattern for the crystalline anhydrous freebase Form I of Compound AI. The crystalline anhydrous freebase Form I exhibited characteristic reflections corresponding to d-spacings of 10.4, 5.5, and 4.1 angstroms. The crystalline anhydrous freebase Form I was further characterized by reflections corresponding to d-spacings of 9.9, 9.2 and 5.0 angstroms. The crystalline anhydrous freebase Form I was even further characterized by reflections corresponding to d-spacings of 3.9, 3.6, and 3.5 angstroms.

In addition to the X-ray powder diffraction patterns described above, the crystalline anhydrous freebase of compound A was further characterized by solid-state carbon-13 nuclear magnetic resonance (NMR) spectra. The solid-state carbon-13 NMR spectra were obtained on a Bruker DSX 500WB NMR system using a Bruker 4 mm H/X/Y CPMAS probe. The carbon-13 NMR spectra utilized proton/carbon-13 cross-polarization magic-angle spinning with variable-amplitude cross polarization, total sideband suppression, and SPINAL decoupling at 100 kHz. The samples were spun at 10.0 kHz, and a total of 1500 scans were collected with a recycle delay of 5 seconds. A line broadening of 10 Hz was applied to the spectra before FT was performed. Chemical shifts are reported on the TMS scale using the carbonyl carbon of glycine (176.03 p.p.m.) as a secondary reference.

The crystalline forms were further characterized by solid state fluorine-19 NMR. The solid-state fluorine-19 NMR spectra were obtained on a Bruker DSX 500WB NMR system using a Bruker 4 mm H/F/X CPMAS probe. The fluorine-19 NMR spectra utilized a simple puse-acquire pulse program. The sample was spun at 15.0 kHz, and a total of 64 scans were collected with a recycle delay of 5 seconds. A line broadening of 10 Hz was applied to the spectrum before FT was performed. Chemical shifts are reported using poly(tetrafluoroethylene) (Teflon®) as an external secondary reference which was assigned a chemical shift of −122 ppm.

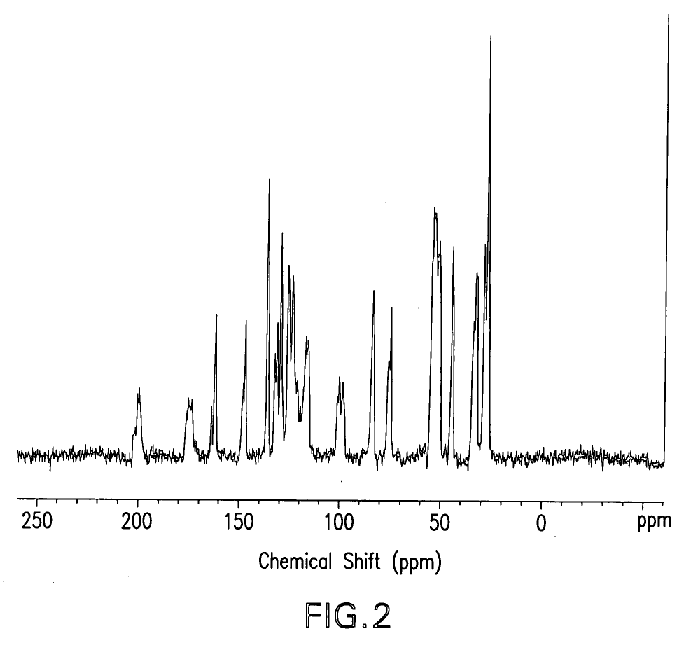

FIG. 2 shows the solid-state carbon-13 CPMAS NMR spectrum for the crystalline anhydrous freebase Form I of compound A. The crystalline anhydrous freebase Form I exhibited characteristic signals with chemical shift values of 27.5, 44.6, and 147.1 p.p.m. Further characteristic of the crystalline anhydrous freebase Form I are the signals with chemical shift values of 32.9, 83.7, and 161.8 p.p.m. The crystalline anhydrous freebase Form I is even further characterized by signals with chemical shift values of 53.9, 75.0, and 136.2 p.p.m.

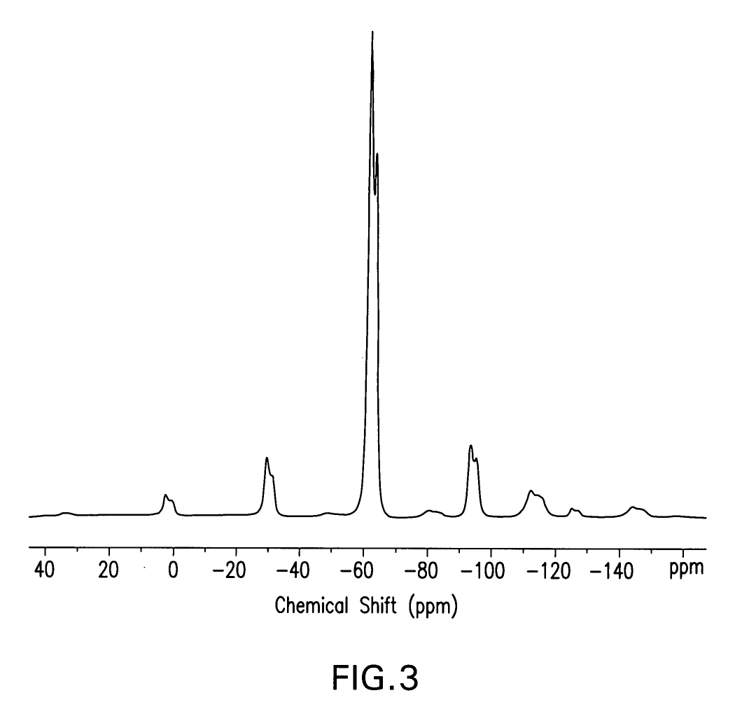

FIG. 3 shows the solid-state fluorine-19 MAS NMR spectrum for the crystalline anhydrous freebase Form I of compound A. The crystalline anhydrous freebase Form I exhibited characteristic signal with chemical shift value of −61.3, −63.1, and −112.2 p.p.m.

DSC data were acquired using TA Instruments DSC 2910 or equivalent instrumentation was used. Between 1 and 6 mg sample was weighed into an open pan. This pan was then placed at the sample position in the calorimeter cell. An empty pan was placed at the reference position. The calorimeter cell was closed and a flow of nitrogen was passed through the cell. The heating program was set to heat the sample at a heating rate of 10° C./min to a temperature of approximately 250° C. The heating program was started. When the run was completed, the data were analyzed using the DSC analysis program contained in the system software. The melting endotherm was integrated between baseline temperature points that are above and below the temperature range over which the endotherm was observed. The data reported are the onset temperature, peak temperature and enthalpy.

Thermogravimetric (TG) data were acquired using a Perkin Elmer model TGA 7 or equivalent instrumentation. Experiments were performed under a flow of nitrogen and using a heating rate of 10° C./min to a maximum temperature of approximately 300° C. After automatically taring the balance, 5 to 20 mg of sample was added to the platinum pan, the furnace was raised, and the heating program started. Weight/temperature data were collected automatically by the instrument. Analysis of the results was carried out by selecting the Delta Y function within the instrument software and choosing the temperatures between which the weight loss was to be calculated. Weight losses are reported up to the onset of decomposition/evaporation.

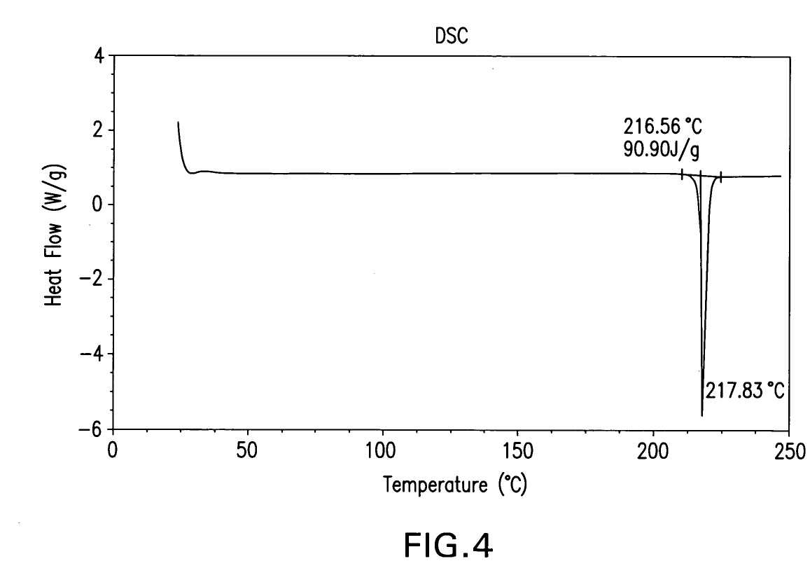

FIG. 4 shows the differential calorimetry scan for the crystalline anhydrous freebase Form I of Compound A. The crystalline anhydrous freebase Form I exhibited an endotherm due to melting with an onset temperature of 216.6° C., a peak temperature of 217.8° C., and an enthalpy change of 90.9 J/g.

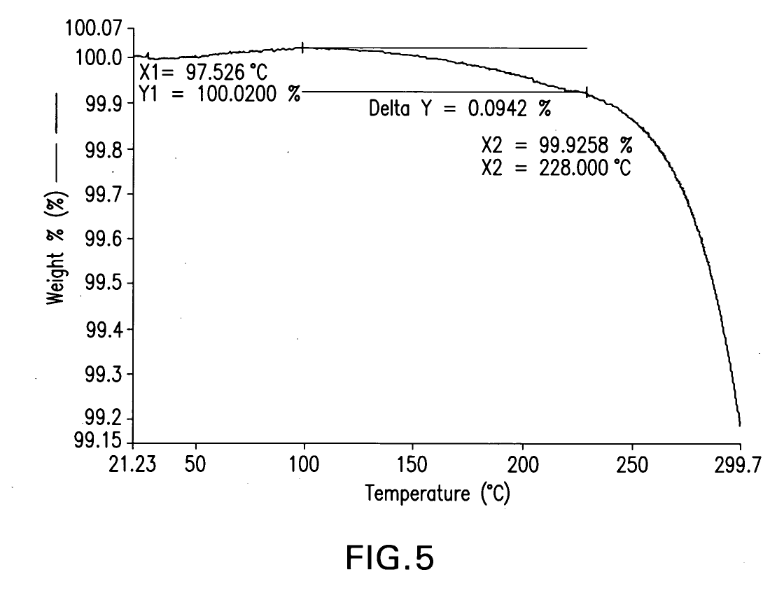

FIG. 5 shows a characteristic thermogravimetric analysis (TGA) curve for the crystalline anhydrous freebase Form I of Compound A. TGA indicated a weight loss of about 0.1% from ambient temperature to about 228° C.

FIG. 6 shows the X-ray diffraction pattern for the crystalline anhydrous freebase Form II of Compound A. The crystalline anhydrous freebase Form II exhibited characteristic reflections corresponding to d-spacings of 7.7, 4.9, and 4.8 angstroms. The crystalline anhydrous freebase was further characterized by reflections corresponding to d-spacings of 5.3, 4.6, and 3.9 angstroms. The crystalline anhydrous freebase Form II was even further characterized by reflections corresponding to d-spacings of 4.2, 3.8, and 2.8 angstroms.

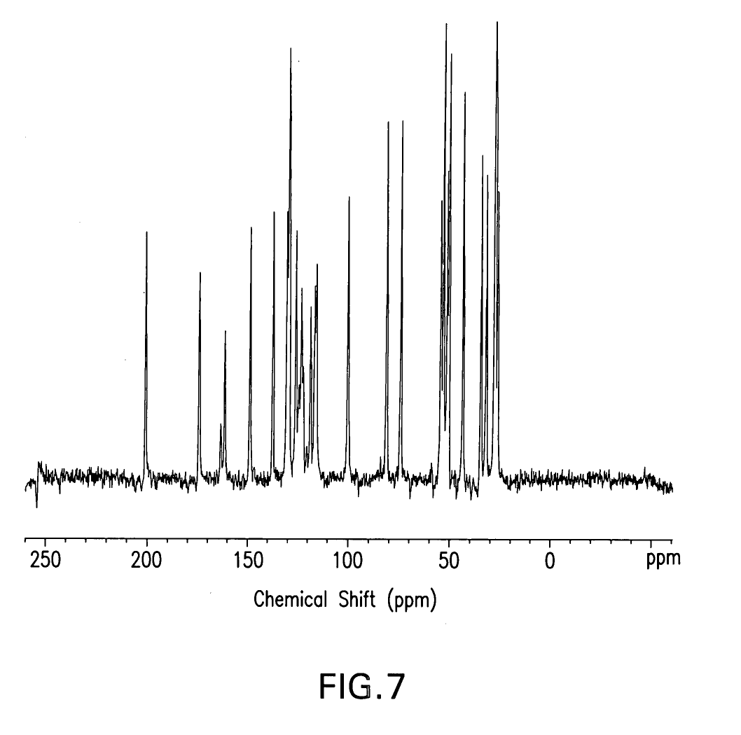

FIG. 7 shows the solid-state carbon-13 CPMAS NMR spectrum for the crystalline anhydrous freebase Form I of compound A. The crystalline anhydrous freebase Form I exhibited characteristic signals with chemical shift values of 28.2, 81.6, and 129.8 p.p.m. Further characteristic of the crystalline anhydrous freebase Form I are the signals with chemical shift values of 74.5, 149.1, and 201.0 p.p.m. The crystalline anhydrous freebase Form I is even further characterized by signals with chemical shift values of 43.7, 100.4, and 129.8 p.p.m.

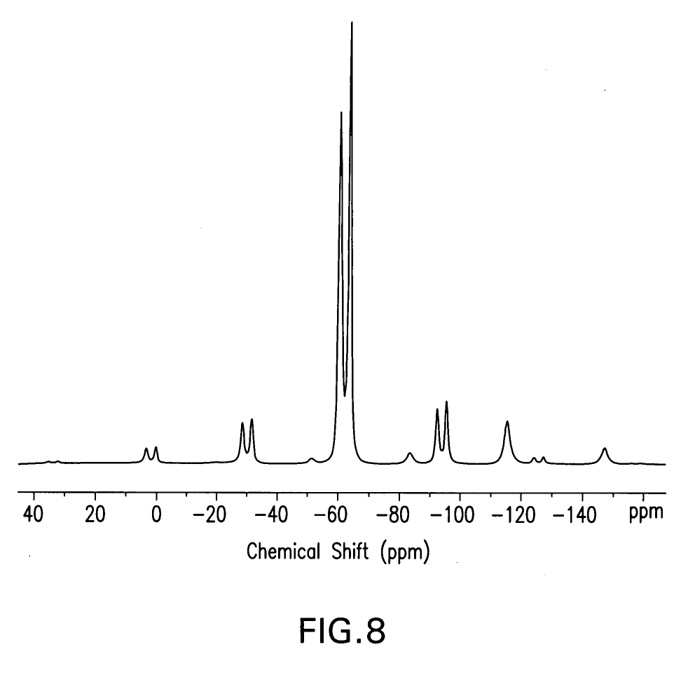

FIG. 8 shows the solid-state fluorine-19 MAS NMR spectrum for the crystalline anhydrous freebase Form II of compound A. The crystalline anhydrous freebase Form II exhibited characteristic signal with chemical shift value of −60.4, −63.4, and −115.3 p.p.m.

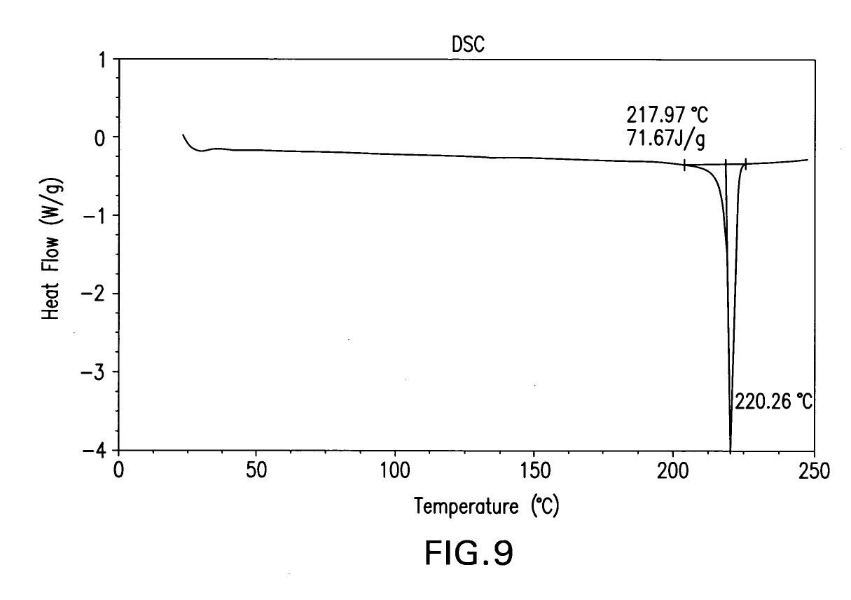

FIG. 9 shows the differential calorimetry scan for the crystalline anhydrous freebase Form II of Compound A. The crystalline anhydrous freebase Form II exhibited an endotherm due to melting with an onset temperature of 218.0° C., a peak temperature of 220.3° C., and an enthalpy change of 71.7 J/g.

The compounds of the present invention are useful in the prevention and treatment of a wide variety of clinical conditions which are characterized by the presence of an excess of tachykinin, in particular substance P, activity. Thus, for example, an excess of tachykinin, and in particular substance P, activity is implicated in a variety of disorders of the central nervous system. Such disorders include mood disorders, such as depression or more particularly depressive disorders, for example, single episodic or recurrent major depressive disorders and dysthymic disorders, or bipolar disorders, for example, bipolar I disorder, bipolar II disorder and cyclothymic disorder; anxiety disorders, such as panic disorder with or without agoraphobia, agoraphobia without history of panic disorder, specific phobias, for example, specific animal phobias, social phobias, obsessive-compulsive disorder, stress disorders including post-traumatic stress disorder and acute stress disorder, and generalized anxiety disorders; schizophrenia and other psychotic disorders, for example, schizophreniform disorders, schizoaffective disorders, delusional disorders, brief psychotic disorders, shared psychotic disorders and psychotic disorders with delusions or hallucinations; delerium, dementia, and amnestic and other cognitive or neurodegenerative disorders, such as Alzheimer's disease, senile dementia, dementia of the Alzheimer's type, vascular dementia, and other dementias, for example, due to HIV disease, head trauma, Parkinson's disease, Huntington's disease, Pick's disease, Creutzfeldt-Jakob disease, or due to multiple aetiologies; Parkinson's disease and other extra-pyramidal movement disorders such as medication-induced movement disorders, for example, neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremour; substance-related disorders arising from the use of alcohol, amphetamines (or amphetamine-like substances) caffeine, cannabis, cocaine, hallucinogens, inhalants and aerosol propellants, nicotine, opioids, phenylglycidine derivatives, sedatives, hypnotics, and anxiolytics, which substance-related disorders include dependence and abuse, intoxication, withdrawal, intoxication delerium, withdrawal delerium, persisting dementia, psychotic disorders, mood disorders, anxiety disorders, sexual dysfunction and sleep disorders; epilepsy; Down's syndrome; demyelinating diseases such as MS and ALS and other neuropathological disorders such as peripheral neuropathy, for example diabetic and chemotherapy-induced neuropathy, and postherpetic neuralgia, trigeminal neuralgia, segmental or intercostal neuralgia and other neuralgias; and cerebral vascular disorders due to acute or chronic cerebrovascular damage such as cerebral infarction, subarachnoid haemorrhage or cerebral oedema.

Tachykinin, and in particular substance P, activity is also involved in nociception and pain. The compounds of the present invention will therefore be of use in the prevention or treatment of diseases and conditions in which pain predominates, including soft tissue and peripheral damage, such as acute trauma, osteoarthritis, rheumatoid arthritis, musculo-skeletal pain, particularly after trauma, spinal pain, myofascial pain syndromes, headache, episiotomy pain, and burns; deep and visceral pain, such as heart pain, muscle pain, eye pain, orofacial pain, for example, odontalgia, abdominal pain, gynaecological pain, for example, dysmenorrhoea, and labour pain; pain associated with nerve and root damage, such as pain associated with peripheral nerve disorders, for example, nerve entrapment and brachial plexus avulsions, amputation, peripheral neuropathies, tic douloureux, atypical facial pain, nerve root damage, and arachnoiditis; pain associated with carcinoma, often referred to as cancer pain; central nervous system pain, such as pain due to spinal cord or brain stem damage; low back pain; sciatica; ankylosing spondylitis, gout; and scar pain.

Tachykinin, and in particular substance P, antagonists may also be of use in the treatment of respiratory diseases, particularly those associated with excess mucus secretion, such as chronic obstructive airways disease, bronchopneumonia, chronic bronchitis, cystic fibrosis and asthma, adult respiratory distress syndrome, and bronchospasm; inflammatory diseases such as inflammatory bowel disease, psoriasis, fibrositis, osteoarthritis, rheumatoid arthritis, pruritis and sunburn; allergies such as eczema and rhinitis; hypersensitivity disorders such as poison ivy; ophthalmic diseases such as conjunctivitis, vernal conjunctivitis, and the like; ophthalmic conditions associated with cell proliferation such as proliferative vitreoretinopathy; cutaneous diseases such as contact dermatitis, atopic dermatitis, urticaria, and other eczematoid dermatitis. Tachykinin, and in particular substance P, antagonists may also be of use in the treatment of neoplasms, including breast tumours, neuroganglioblastomas and small cell carcinomas such as small cell lung cancer.

Tachykinin, and in particular substance P, antagonists may also be of use in the treatment of gastrointestinal (GI) disorders, including inflammatory disorders and diseases of the GI tract such as gastritis, gastroduodenal ulcers, gastric carcinomas, gastric lymphomas, disorders associated with the neuronal control of viscera, ulcerative colitis, Crohn's disease, irritable bowel syndrome and emesis, including acute, delayed or anticipatory emesis such as emesis induced by chemotherapy, radiation, toxins, viral or bacterial infections, pregnancy, vestibular disorders, for example, motion sickness, vertigo, dizziness and Meniere's disease, surgery, migraine, variations in intercranial pressure, gastro-oesophageal reflux disease, acid indigestion, over indulgence in food or drink, acid stomach, waterbrash or regurgitation, heartburn, for example, episodic, nocturnal or meal-induced heartburn, and dyspepsia.

Tachykinin, and in particular substance P, antagonists may also be of use in the treatment of a variety of other conditions including stress related somatic disorders; reflex sympathetic dystrophy such as shoulder/hand syndrome; adverse immunological reactions such as rejection of transplanted tissues and disorders related to immune enhancement or suppression such as systemic lupus erythematosus; plasma extravasation resulting from cytokine chemotherapy, disorders of bladder function such as cystitis, bladder detrusor hyper-reflexia, frequent urination and urinary incontinence, including the prevention or treatment of overactive bladder with symptoms of urge urinary incontinence, urgency, and frequency; fibrosing and collagen diseases such as scleroderma and eosinophilic fascioliasis; disorders of blood flow caused by vasodilation and vasospastic diseases such as angina, vascular headache, migraine and Reynaud's disease; and pain or nociception attributable to or associated with any of the foregoing conditions, especially the transmission of pain in migraine. The compounds of the present invention are also of value in the treatment of a combination of the above conditions, in particular in the treatment of combined post-operative pain and post-operative nausea and vomiting.

The compounds of the present invention are particularly useful in the prevention or treatment of emesis, including acute, delayed or anticipatory emesis, such as emesis induced by chemotherapy, radiation, toxins, pregnancy, vestibular disorders, motion, surgery, migraine, and variations in intercranial pressure. For example, the compounds of the present invention are of use optionally in combination with other antiemetic agents for the prevention of acute and delayed nausea and vomiting associated with initial and repeat courses of moderate or highly emetogenic cancer chemotherapy, including high-dose cisplatin. Most especially, the compounds of the present invention are of use in the treatment of emesis induced by antineoplastic (cytotoxic) agents, including those routinely used in cancer chemotherapy, and emesis induced by other pharmacological agents, for example, rolipram. Examples of such chemotherapeutic agents include alkylating agents, for example, ethyleneimine compounds, alkyl sulphonates and other compounds with an alkylating action such as nitrosoureas, cisplatin and dacarbazine; antimetabolites, for example, folic acid, purine or pyrimidine antagonists; mitotic inhibitors, for example, vinca alkaloids and derivatives of podophyllotoxin; and cytotoxic antibiotics. Particular examples of chemotherapeutic agents are described, for instance, by D. J. Stewart in Nausea and Vomiting: Recent Research and Clinical Advances, Eds. J. Kucharczyk et al, CRC Press Inc., Boca Raton, Fla., USA (1991) pages 177-203, especially page 188. Commonly used chemotherapeutic agents include cisplatin, dacarbazine (DTIC), dactinomycin, mechlorethamine, streptozocin, cyclophosphamide, carmustine (BCNU), lomustine (CCNU), doxorubicin (adriamycin), daunorubicin, procarbazine, mitomycin, cytarabine, etoposide, methotrexate, 5-fluorouracil, vinblastine, vincristine, bleomycin and chlorambucil [R. J. Gralla et al in Cancer Treatment Reports (1984) 68(1), 163-172]. A further aspect of the present invention comprises the use of a compound of the present invention for achieving a chronobiologic (circadian rhythm phase-shifting) effect and alleviating circadian rhythm disorders in a mammal. The present invention is further directed to the use of a compound of the present invention for blocking the phase-shifting effects of light in a mammal.

### PREPARATORY EXAMPLE 1

### Process to Ketone

### Step 1: 2-(4-Fluorophenyl)-N-methoxy-N-methylacetamide (2)

### Summary:

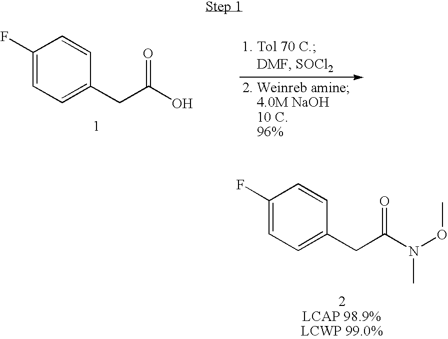

This reaction gives consistently high yield and high purity of material. No major side products have been identified. The final product is an oil (typically clear or slightly yellow) and is isolated with the above purity profile from the crude work up.

### Procedure:

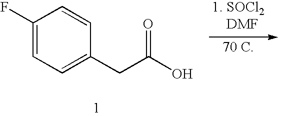

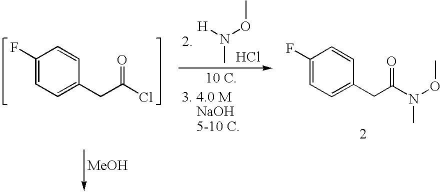

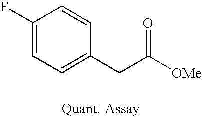

| | FW: | Amt. | Moles | Equiv. | |
|---|---|---|---|---|---|
| 4- | 154 | 5.0 kg | 32.47 mol | 1.0 eq. | |
| Fluorophenyl- | | | | | |
| acetic acid (1) | | | | | |
| DMF | 73.1 | 48 mL | 0.65 mol | 0.02 eq. | |
| SOCl₂ | 119 | 2.84 L | 38.96 mol | 1.2 eq. | Weinreb |
| amine-HCl | 97.5 | 4.75 kg | 48.70 mol | 1.5 eq. | |
| NaOH | 4.0 M | 32.47 L | 129.87 mol | 4.0 eq. | |
| Toluene | — | 49.19 L- | | | |
| Brine | — | 64.92 L- | | | |

A 100 L extractor equipped with a reflux condenser, and a base scrubber was charged with toluene (49.2 L, KF≦100 ppm) and 4-fluorophenylacetic acid (1) was added (5.0 kg). This solution was heated to 70° C. Once 70° C. was reached the DMF (48 mL, KF≦150 ppm) was added and thionyl chloride (2.8 L) was slowly added over 3 hours.

Batch temperature will decrease while thionyl chloride is added. Typical temperature changes range from 6-10° C.

When all thionyl chloride has been added and off-gassing has ceased (typically 30 min. after addition is complete) an aliquot of the batch was quenched into excess methanol for HPLC analysis as the methyl ester.

Reaction is done when acid 1 is at <0.5 LCAP.

Next the reaction was cooled to 5-10° C. The Weinreb amine-HCl (4.75 kg) was added to the batch at this point. Slow addition of NaOH (32.5 L) was begun at this point. This base was added at a rate that maintained the batch temperature at or below 10° C. with a typical addition time of 3 hours. Once this addition was done an aliquot of the batch was quenched into MeOH and assayed by HPLC to check for complete consumption of the acid chloride.

Complete consumption of the acid chloride (in the form of the methyl ester after this quench) should be seen. Additional base can be added if the acid chloride is still present.

The biphasic solution was separated at between 5° C. and room temperature and the organic phase was washed with 15 wt. % NaCl (aq) (2×32.5 L).

Typical assay yield of the organic phase was 96%.

The organic phase was concentrated to a 50 wt. % solution (typical KF≦500 ppm).

### Step 2: 1-(4-Fluorophenyl)but-3-en-2-one (3)

### Summary:

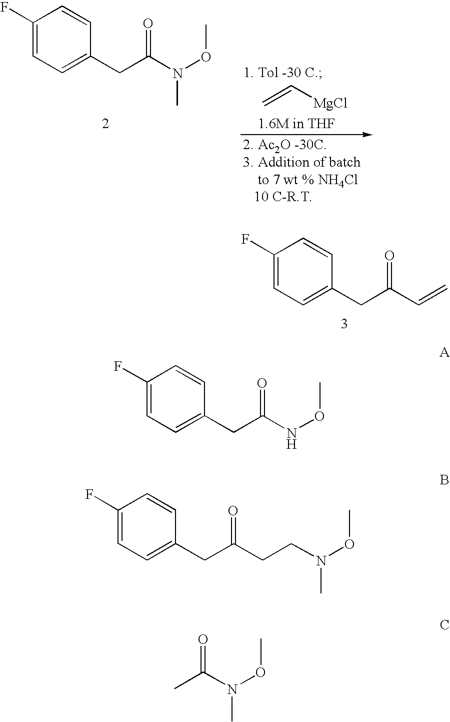

This reaction is very sensitive to the quality of the Grignard reagent and the quench method. Major side products have been identified (A, B, C), and are shown above. The product is unstable when concentrated to an oil, and has moderate stability in solution. The final toluene solution should be kept cold and used in the next step without delay.

### Procedure:

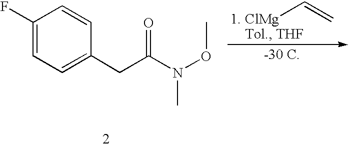

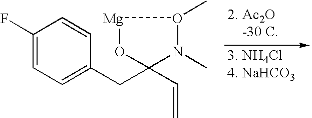

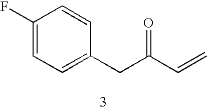

| | FW: | Amt. | Moles | Equiv. |
|---|---|---|---|---|
| Weinreb amide (2) | 197.2 | 157.2 g | 0.800 mol | 1.0 eq. |
| Vinyl magnesium | 1.6 M | 700 mL | 1.12 mol | 1.4 eq. |
| chloride in THF | | | | |
| Ac₂O (density | 102 | 151 mL | 1.60 mol | 2.0 eq. |
| 1.082 g/mL) | | | | |
| NH₄Cl (2.5 wt % aq. | | 1.29 L | — | — |
| solution) | | | | |
| Toluene (density | | | | |
| 0.865 g/mL) | | −1.54 L | — | — |

A 3 L round bottom flask equipped with an addition funnel was charged with the Weinreb amide 2 as a 61% wt solution in toluene (262 g tot mass; 157.2 g 2, 105 g toluene). This solution was diluted to a 0.5 M solution of amide 2 in toluene by addition of 1.32 L of toluene (KF of solution <150 ppm). This solution was cooled to −30° C., and vinyl magnesium chloride was slowly added.

During the addition of vinyl magnesium chloride the batch temperature is maintained at −30° C. Typical addition time is around 60 minutes.

After the vinyl Grignard addition was complete the reaction was allowed to age at −30° C. for 60 minutes. The reaction was checked by HPLC after this 60 minute age.

Acetic anhydride (151 mL) was then slowly added to the reaction.

Batch temperature is maintained at −30° C. during this addition to avoid impurities. Typical time is 30 minutes. Assay of the reaction at the end of this addition typically shows approximately 0.5% LCAP of impurity B when compared to product.

In a separate 5 L 3-neck round bottom flask a 2.5 wt % solution of NH₄Cl in water (1.29 L) was cooled to 10° C. The batch at −30° C. was cannulated to this vigorously stirred ammonium chloride solution.

The final temperature of the batch is typically around 12-13° C.

When the batch had reached ambient temperature the aqueous and organic layers were cut. The organic layer was then washed with water (1.3 L). The organic layer was dried with MgSO₄ powder (˜100-200 g) until the KF of this solution reached at or below 1000 ppm. The solids were filtered away and washed with dry MeCN (4×50 mL) to provide a solution of the product in THF/MeCN/Toluene (˜2.0 L, KF ˜970 ppm, 1.80 kg, 7.29 wt %, 131 g of 3, 100% yield) which was used directly in the next step.

The impurity profile shows 1.5 LCAP of impurity B and 9.1 LCAP of impurity C.

### Step 3: TES dienyl ether

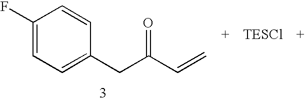

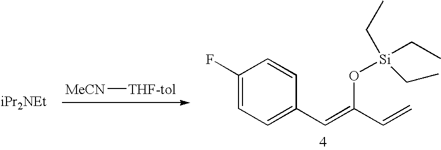

| Materials: | FW | amount | mmol | equiv. |
|---|---|---|---|---|
| vinyl ketone 3 | 164.18 | 6.62 g | 40.3 | 1.0 |
| TESCl | 150.73 | 9.72 g | 64.5 | 1.6 |
| DIPEA | 129.25 | 8.34 g | 64.5 | 1.6 |
| MeCl | | 18 mL | | |
| NH₄CN (2.0 wt % aq) | | 70 mL | | |
| Toluene | | 100 mL | | |

### Procedure:

To 90.8 g of the 7.29 wt % enone 3 solution in THF/MeCN/toluene obtained from step 2 at rt was added more dry MeCN (18 mL) and iPr₂NEt. TESCl was then added slowly while maintaining rt. The solution was stirred at rt until LC revealed complete conversion (˜16 h).

The reaction was quenched with 2 wt % aq NH₄Cl (70 mL). The organic layer was separated and washed with water (70 mL). It was then concentrated and flushed with toluene to ˜37 wt % with a KF of ˜200 ppm. Assay yield: 8.64 g, 77%. NMR shows <5% of the E-isomer.

### Step 4: Di-(−)-menthylfumarate

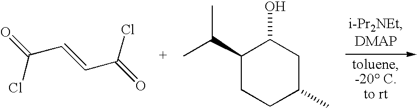

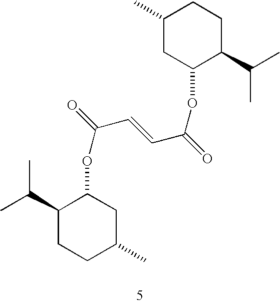

| Materials: | FW | d/conc | amount | mol | equiv. |
|---|---|---|---|---|---|
| fumaryl chloride | 152.96 | 1.412 | 80.5 g (95%) | 0.50 | 1.0 |
| (1R,2S,5R)-(−)- | 156.27 | | 157.8 g | 1.01 | 2.02 |
| menthol | | | | | |
| i-Pr₂NEt | 129.25 | 0.742 | 191 mL | 1.10 | 2.20 |
| DMAP | 122.17 | | 3.05 g | 0.025 | 0.05 |
| toluene | | | 600 mL | | |
| 1.0 N HCl | | | 110 mL | 0.11 | 0.22 |
| NaCl | | | 60 g | | |

### Procedure:

To a 3-neck flask was charged toluene (500 mL) and (−)-menthol (157.8 g). The solution was cooled to −20° C. and fumaryl chloride (80.5 g of 95%) was charged with 80 mL toluene flush (no exotherm was observed). i-Pr₂NEt (191 mL) was added over 30 min (fuming) with 20 mL toluene flush at −20° C. DMAP was added immediately afterwards. The dark slurry was then allowed to warm to 21° C. over ˜60 min to give a dark solution, which showed complete conversion by HPLC.

At room temperature, a mild exotherm caused the temperature to rise to ˜30° C. It will be desirable to age at −20 to 0° C. for 1-2 h before warming up to rt.

600 mL of aqueous 3% NaCl was added. The aqueous layer (˜800 mL) was cut away and the organic layer was washed with 800 mL aqueous 0.15 N HCl containing 5 wt % NaCl. The dark organic layer (˜800 mL, 710 g) showed 93% assay yield (182 g, 0.464 mol product in 98LCAP) and was concentrated to 378 g (48 wt %) for direct use in the Diels-Alder reaction. ¹H NMR showed non-detectable menthol.

### Step 5: Diels-Alder

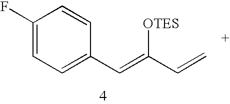

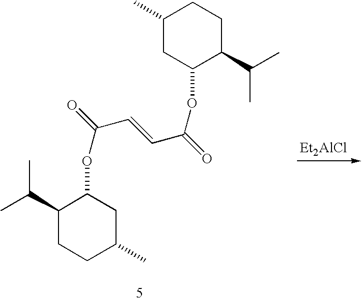

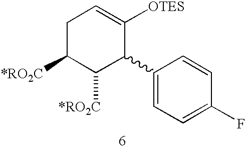

| Materials: | FW | amount | mmol | equiv. |
|---|---|---|---|---|
| diene 4 | 278.44 | 8.64 g | 31 | 1.0 |
| di-(−)-menthyl fumarate 5 | 392.57 | 14.6 g | 37.2 | 1.2 |
| diethylaluminum chloride | 1.8 M/tol | 29.3 mL | 52.7 | 1.7 |
| hydrochloride acid | 3.0 N | 52.7 mL | 158.1 | 5.1 |
| hydrochloric acid | 1.0 N | 100 mL | 100 | 3.2 |
| NaOH | 0.5 N | 50 mL | 25 | 0.81 |

### Procedure:

The toluene solutions of diene 4 (23.4 g, 37 wt %) and dimenthylfumarate 5 (30.4 g, 48 wt %) were combined and cooled to 0° C. Diethylaluminum chloride solution in toluene (1.8 M, 29.3 mL) was added over 45 min, keeping the temperature below 5° C. (exothermic addition). The dark orange solution was aged at 0° C. for 18 h (˜90% conversion), and then at 21° C. for 6 h when it reached >95% conversion.

If the desired conversion was not achieved, more Lewis acid (and dimenthyl fumarate if necessary) could be added at any point of the reaction.

The reaction mixture was carefully quenched with aqueous 3 N HCl (8 mL) over >60 min while keeping the temperature at 15-25° C.

It is important to add this first portion of HCl very slowly without any bursts. Although the batch is not very sensitive to heat, rapid off-gassing and foaming upon addition of HCl could result in a disastrous overflow of the batch. The foaming needs to be watched very closely.

The remaining HCl (3N, 44.7 mL) was added slowly while keeping temperature at 15-25° C., and the resulting mixture was aged for 30 min at rt. The aqueous layer was removed, and the organic layer was washed with 1 N aq HCl (2×50 mL) and 0.5 N aq NaOH (50 mL). The toluene solution was used directly in the next step

Any E-isomer of the diene (<5%) which is present does not react in the Diels-Alder reaction. A small amount of deprotected products 7 could form in the organic layer during the work-up.

### Step 6: Deprotection and Epimerization

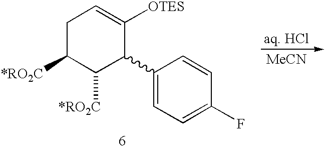

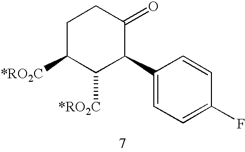

| Materials: | FW | amount | mmol | equiv. |
|---|---|---|---|---|
| cyclohexane 6 | 671.01 | | 31 | 1.0 |
| acetonitrile | | 211 mL | | |
| aqueous 6.0 N HCl | | 6.2 mL | 37.3 | 1.2 |

R* = (−)-menthyl

### Procedure:

The toluene solution from step 5 was concentrated to remove all solvents, flushed with acetonitrile, to give 210 mL slurry in acetonitrile. Aqueous 6 N HCl (6.2 mL) was added. The slurry was stirred at room temperature for ˜2 h, at which point HPLC indicated that the reaction was complete.

The desilylation initially gave a mixture of 2,3-cis and 2,3-trans ketones, which, driven by crystallization of desired 7, isomerized to predominantly trans.

After aging, filtration followed by 3×51.4 mL (3.5 volumes) acetonitrile slurry washes and drying in vacuo overnight at 60° C. yields a white solid (15.3 g, 98.6 wt %, 87% yield).

### PREPARATORY EXAMPLE 2

### Ketone Reduction to Compound A

### Step 1. Ketone Reduction to Triol

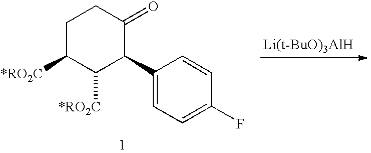

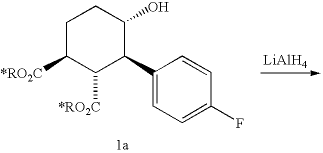

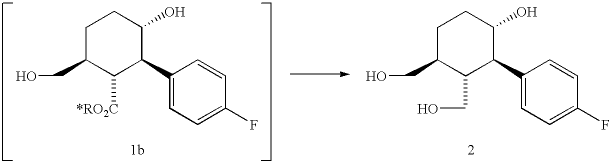

| Materials: | FW | mass | volume | mol | | equiv. |
|---|---|---|---|---|---|---|
| ketone 1 | 556.75 | 7.25 kg (96.5 wt %) | | | 12.57 | 1 |
| Li(t-BuO)₃AlH (1.0 M THF) | | | 18.9 | L | 18.86 | 1.5 |
| THF (KF = 74.5 ppm) | | | 25 | L | | |
| LiAlH₄ (2.3 M THF) | | | 7.11 | L | 16.34 | 1.3 |
| HCl (50% v/v, 6.0 N) | | | 29.5 | L | 177 | 14.1 |
| HCl (conc., 12 N) | | | 8.9 | L | 106.8 | 8.5 |
| Heptane | | | 35 | L | | |
| NaOH (10.0 N) | | | 14 | L | 140 | 11.1 |
| Ethyl Acetate | | | 2 × 39 | L | | |
| 2,4,6-collidine | 121.18 | 0.064 kg | 2 × 0.035 | L | 2 × 0.25 | 0.04 |

R* = (−)-menthyl

### Procedure (179553-01)

Add 22 L THF to a 100 L RBF with an inert atmosphere. Cool the flask to −40° C. and add Li(O-tBu)₃AlH. Charge ketone 1 as a solid with a THF (3 L) rinse while keeping temp <−25° C. Stir at −30 to −35° C. until <5% starting material remains (all solid dissolves), approximately 2-3 hours. The trans/cis ratio is typically ˜25.

Warm the reaction mixture to ˜−20° C. and add LiAlH₄. Allow the batch warm up to ˜10° C. and apply cooling to keep the temperature <30° C. Stir the reaction mixture at room temperature until observing complete reduction to the triol (<0.5% desired diol 1 b left), >3 hours.

Cool the batch to ˜0° C. and reverse quench slowly into 6.0 N HCl (23.5 L) while keeping the temperature <40° C. Use 2 L THF to rinse the reaction vessel. Caution! Significant H₂ off-gassing and exotherm will occur over the entirety. Two clear layers should form if settling occurs. Concentrate the quenched solution to ˜30 L (4.3V) (water starts to condense at this point).

Add heptane (35 L) followed by 6.0 L 6.0 N HCl and 8.9 L 12.0N HCl to dissolve a rag layer. Cut and keep the aqueous layer (˜40 L) (org layer ˜43 L), being certain to keep any rag (<250 mL) with the aqueous. Assay each layer to ascertain the menthol distribution, which should show <2% remaining in the aqueous. Charge the aq layer back to the extractor with 1 L water rinse. Titrate to pH ˜1.5-2 with ˜14 L 10N NaOH while keeping temperature <30° C. (charge 12 L first, followed by 0.5 L portions; pH is ˜0 after 13 L is charged; it could take ˜10-15 min for pH meter to give a stable pH reading.).

Add 39 L EtOAc and stir vigorously for 30 min. Make sure pH is ˜1.5-2, otherwise add 10 N NaOH or conc HCl in 250 mL portions to adjust the pH. Allow 1-2 h for the emulsion layer to break up. Cut and keep the aqueous (50 L), which should show ˜14% product remaining. Drum off the organic (41 L) followed by addition of collidine (35 mL) to adjust to pH ˜4-4.5. Repeat the extraction once more with 39 L EtOAc (faster settling this time). The aqueous layer should show ˜2% product remaining and is discarded.

A pH of −0.4 would result in slow decomposition of the triol, possible to acetate at ˜0.1%/h. A higher pH to ˜1.8-2.0 reduces the aq solubility of triol, but too high a pH would result in gel formation (Al(OH)₃?). The triol solution in EtOAc is stable at pH 1.4-5 at rt and at pH 4-5 at 45° (8 days).

Concentrate the combined organic layers and flush with EtOAc to ˜9 L with a KF<1000 ppm. Drum off with an inline filter with 3 L MeCN rinse. Expected yield: 2.91 kg of trans-triol (91% Y), 3.02 kg total triols (trans/cis ˜25). The resulting solution is stable at rt for >9 days and at 50° C. for >4 days.

### Step 2: Sulfonylation

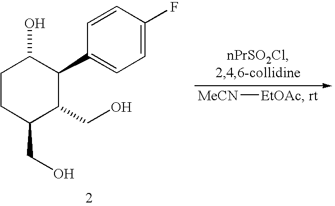

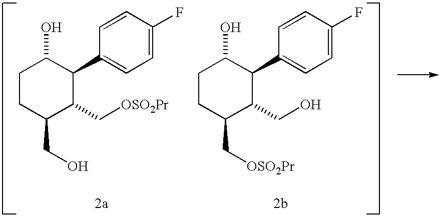

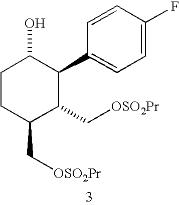

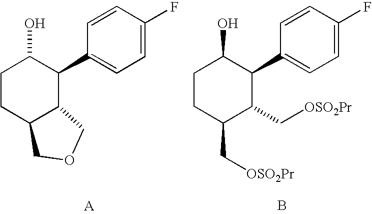

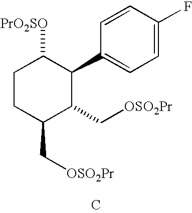

| Materials: | FW | mass | volume | mol | equiv. |
|---|---|---|---|---|---|
| Triol 2 | 254.30 | 4.34 kg | | 17.1 | 1.0 |
| nPrSO₂Cl | 142.6 | 6.59 kg | 5.18 L | 46.2 | 2.7 |
| collidine | 121.18 | 6.01 kg | 6.55 L | 49.6 | 2.9 |
| MeCN | | | 22 L | | |
| HCl (1.0 N) | | | 21.6 L | 21.6 | 1.26 |
| EtOAc | | | 14 L | | |
| NaCl 10% aq | | | 38 L | | |
| HCl (50% v/v) | | | 0.50 L | 3 | 0.018 |
| NaOH (1.0 N) | | | 30 L | 30 | 1.75 |
| NaCl 6% aq | | | 20 L | | |

### Procedure (179553-17):

Charge the triol solution (containing 4.34 kg active triol+0.23 kg of other triols and ˜8.7 L EtOAc+4 L MeCN, KF ˜2000 ppm, equiv 10 mol % H₂O), MeCN (14 L) and n-PrSO₂Cl to a 100 L extractor. Cool the solution to 15° C. and add collidine all in one portion. Apply cooling to keep the reaction temperature at 18-21° C. A slurry forms within 30 min.

Monitor the reaction by LC every hour after 2 h mark until no starting material and <2.5% of the mono-sulfonates 2a+b are left (typically 4-6 hours). Leaving the reaction run for longer leads to more tri-sulfonate C formation.

After 230 min (2a+b: 120 min—14.4 A %, 180 min—4.6 A %, 210 min—1.4 A %, non-SM related peaks-collidine, EtOAc, n-PrSO₂Cl—are not integrated), quench the reaction with 1 N HCl (21.6 L) and add 14 L more EtOAc The quench is slightly endothermic to ˜15° C. and then back to ˜18° C. Cut away the bottom aqueous layer (˜34 L). Wash the organic layer with 10% NaCl (38 L) combined with 50% v/v HCl (6.0 N, 0.50 L) to remove any residual collidine. Cut away the bottom aq layer (˜41 L) and add NaOH (1 N, 30 L, removing PrSO₂Cl) to the organic layer while keeping temperature <27° C. Stir for 15 min and let the layers settle. Cut away the aq layer (˜36 L) and wash the organic layer, which should show <2 mol % of n-PrSO₂Cl left, with 6% NaCl (20 L). Cut away the aq layer (˜24 L) and collect the organic layer (25.6 kg) with 1 L EtOAc rinse and assay for yield (6.80 kg 3, 85%).

It is then concentrated to an oil, flushed with 20 L cyclohexane to an oil and then with 30 L CH₂Cl₂ to ˜10 L (transfer the solution to a new flask via an inline filter after 15 L CH₂Cl₂ is used and then continue the distillation), when KF should be <250 ppm and EtOAc <8 mol % by LC.

The solution is used for the next reaction.

### Step 3: Imidate Preparation

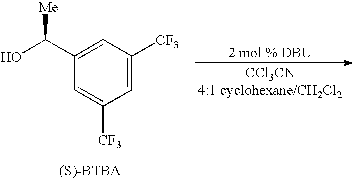

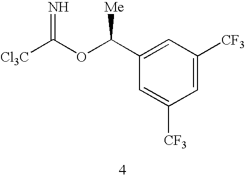

| Materials: | FW | mass | volume | mol | equiv. |
|---|---|---|---|---|---|
| (S)-BTBA | 258.16 | 8.00 kg | | 31.0 | 1.0 |
| CCl3CN | 144.4 | 4.92 kg | 3.42 L | 34.07 | 1.1 |
| DBU | 152.24 | | 92.2 mL | 0.62 | 0.02 |
| Cyclohexane | | | 42.4 L | | |
| CH₂Cl₂ | | | 8.6 L | | |

### Procedure:

To a 100 Liter Flask containing 27 L of a 4:1 mixture of cyclohexane/CH₂Cl₂ was added 8.0 Kg of(S)-BTBA as a solid and the sides of the flask were rinsed with an additional 10.3 L of 4:1 mixture of cyclohexane/CH₂Cl₂. To the resulting slurry was added 4.92 Kg (3.42 Liters) of trichloroacetonitrile followed by 92.2 mL of DBU. The reaction mixture was aged at rt for 5.5 h and assayed for completion. The reaction mixture was then transferred to a 100 Liter extractor rinsing the reaction flask with cyclohexane. The mixture was washed with 27 Liters of water and then with 27 liters of brine. The organic layer was then filtered over a small plug of Solka floc and azetropically distilled under reduced pressure (24 mmHg, internal temp<35° C.) and a final volume of ˜15 Liters and a Kf<200. Assay yield=12.00 Kg (96.2%).

### Step 4: Etherification

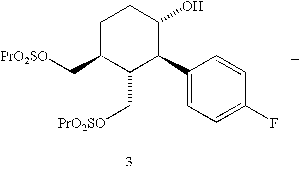

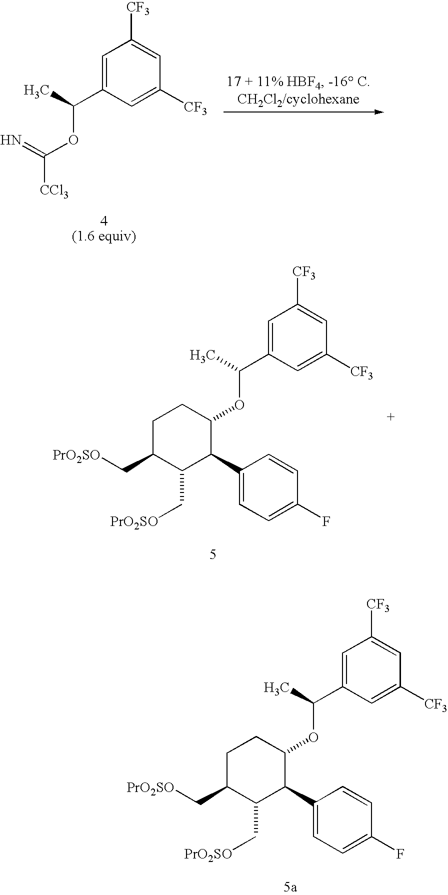

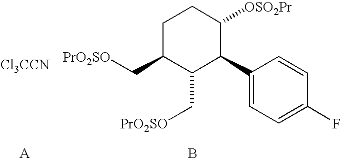

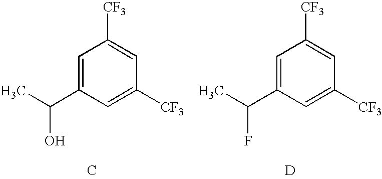

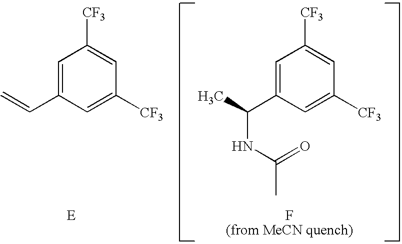

| Materials: | FW | mass | volume | mol | equiv. |
|---|---|---|---|---|---|
| Alcohol 3 | 466.58 | 6.73 kg | | 14.4 | 1.0 |
| Imidate 4 | 402.55 | 9.33 kg | | 23.2 | 1.6 |
| | | active | | | |
| HBF₄ (54 wt % in Et₂O) | 87.8 | | 0.558 L | 4.09 | 0.28 |
| cyclohexane | | | ~10 L | | |
| CH₂Cl₂ | | | ~9 L | | |
| NaOH (2.0 N) | | | 16 L | 32 | 2.2 |
| IPA | | | 125 L | | |

### Procedure:

Charge the CH₂Cl₂ solution of the cyclohexanol 3 (containing 6.73 kg active 3+˜0.78 kg of related other alcohols and ˜6 L CH₂Cl₂, KF <250 ppm, equiv <1.2 mol % H₂O) to a 100 L extractor. Charge the imidate solution (˜850 g/L in cyclohexane, ˜11 L, containing ˜2 L cyclohexane) followed by additional cyclohexane (8.0 L). The mixture turns cloudy due to 3 oiling out. Add more CH₂Cl₂ (2 L) to dissolve the oil. Cool to −17° C. (oiling out at ˜0° C.) and add more CH₂CL₂ (1.3 L) to dissolve the oil. The KF at this point should be <110 ppm (<1.5 mol % water). Add 0.17 equiv of HBF₄ (0.339 L) in one portion, resulting in temperature rising to −16° C. The slightly cloudy mixture is aged at −16° C. It turns clear in ˜40 min and a slurry starts to form and thickens as the reaction proceeds to generate poorly soluble trichloroacetamide A.

After aging at −16° C. for 18 hours, LC assay reveals ˜82% conv and a 5/5a ratio of ˜6. For slightly higher conversion, 0.11 equiv more HBF₄ (0.219 L) is added following by aging at −16° C. for 4 h. The reaction is then warmed to 5° C. and aged for 1 h before being quenched with NaOH (2 N, 16 L). The exotherm brings the temperature to 18° C. After aging at rt for ˜15 min, the layers are allowed to settle. The bottom aqueous layer (˜18 L) is cut away and the organic layer is washed with 18 L of water. The cloudy bottom organic layer is collected (assay yield of 5: ˜74%), concentrated to ˜20 L, and flushed with IPA (90 L) while keeping batch temperature at ˜40° C. and volume at ˜50-60 L to enable stirring as the product crystallizes out. A final volume of ˜70 L is reached and the thick slurry is aged at rt until mother liquor shows <11 g/L loss (5/5a <0.55). The product is then filtered, washed with IPA (35 L), and dried. 7.07 kg, 98 A %, 96 wt %, 6.82 kg corrected, 67% yield.

### Step 5: Cyclization with Allylamine

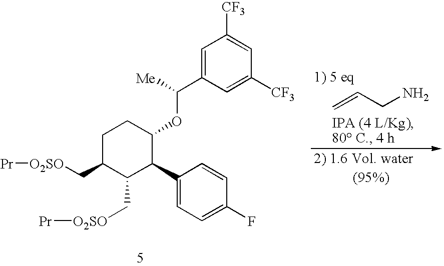

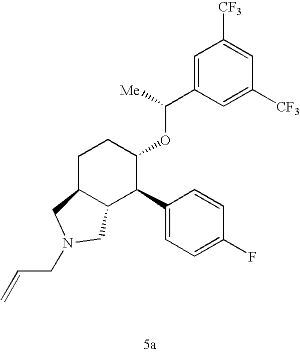

| Materials: | FW | mass | volume | mol | equiv. |
|---|---|---|---|---|---|
| Bis-propylsulfonate 5 | 706.73 | 6.79 kg | | 9.61 | 1.0 |
| allylamine | 57.09 | 2.85 kg | 3.74 L | 48.0 | 5.0 |
| IPA | | | 27.0 L | | |
| Water | | | 43.0 L | | |

### Procedure:

The reaction vessel was charged with IPA (27 L), allylamine (3.74 L, 50.0 moles), and bis-propylsulfonate (6.79 kg, 9.61 moles).

At room temperature, the mixture was a very thick (pasty) mixture that was difficult to stir. The reaction mixture loosens up upon heating and became completely homogeneous at +55-60° C. Note that allylamine was boiling at +53° C.

The mixture was heated to +75-80° C. for 4 h, and was cooled to +40° C. to room temperature. One half volume of water (13.5 L) was added and the batch was seeded (ca. 35 g, 0.5 wt %).

The batch may crystallize without seed but seeding gave more consistent results.

The batch was aged for 30 min and the remainder of water (29.5 L) was added over a couple hours. It was filtered, washed with 65/35 H2O/IPA (12 L). Product was dried at +40° C. for 24 hours under a stream of nitrogen to give 4.9 Kg of product (95% yield).

### Step 6: Deprotection

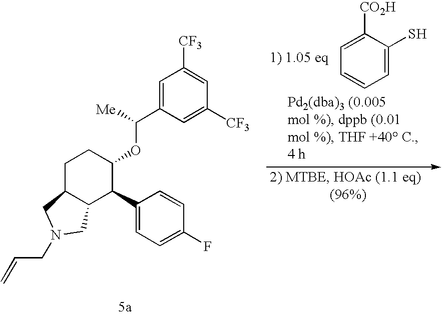

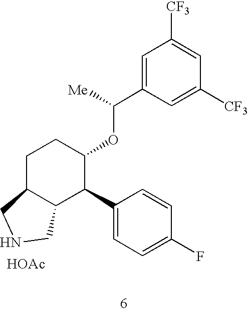

| Materials: | FW | mass | volume | mol | equiv. |
|---|---|---|---|---|---|
| Allyl-protected | 515.5 | 5.16 kg | | 10.0 | 1.0 |
| pyrrolide | | | | | |
| THF | | | 25.8 L | | |
| Thiosalicyclic | 154.2 | 1.62 kg | | 10.5 | 1.05 |
| acid | | | | | |
| Pd₂(dba)₃ | 915.7 | 46.0 g | | 0.005 | 0.005 |
| Dppb | 426.5 | 43.0 g | | 0.01 | 0.01 |
| MTBE | | | 41.0 L | | |
| NaOH | | | 25.8 L (1 N) | 25.8 | |
| Water | | | 46.0 L | | |
| AcOH | 60.05 | 660 g | 629 mL | 11.0 | 1.1 |

Procedure:

The reaction vessel was charged with THF (25.8 L), allylamine protected pyrrolidine (5.16 Kg, 10.0 moles), and thiosalicylic acid (1.62 Kg, 10.5 moles). The reaction mixture was degassed and dppb (4.3 g, 0.01 mol) was added followed by Pd₂(dba)₃ (4.6 g, 0.005 mol) under nitrogen.

The mixture was stirred at +40° C. for 4 h, cooled to r.t and was reverse added into a stirred biphasic mixture made of MTBE (41 L) and 1 N aqueous NaOH solution (25.8 L). Layers were separated and the organic was washed with water (2×23 L). The organic solution was concentrated under vacuum with feeding of MTBE (in-line filtration) with a constant volume of ca. 45 L to lower the KF to less than 5000 ppm.

THF at the end of distillation is ≦10 vol %.

The mixture (ca. 8-10 L MTBE/Kg) is heated to ca. +50° C. and acetic acid (10 Vol %, 62.9 mL) was added and the batch was seeded (0.1 wt %, 5 g) to initiate the crystallization. It was aged at +50° C. for 30 minutes and remaining acetic acid (535.5 mL) was added over ca. 1 h at +50° C.

The salt crystallizes as a quite thick slurry but remains stirrable. It loosens up upon aging. Alternatively, acetic acid can be added as an MTBE solution (ca. 1 M).

After aging at +50° C. for 2 h the batch was cooled to room temperature and aged for another 2 h, it was filtered, washed with MTBE (8 L) and dried at +40° C. under vacuum for 24 h to give 5.14 Kg of the product (96% yield). Pd was ca. 25 ppm.

### Step 7: Preparation of Compound A

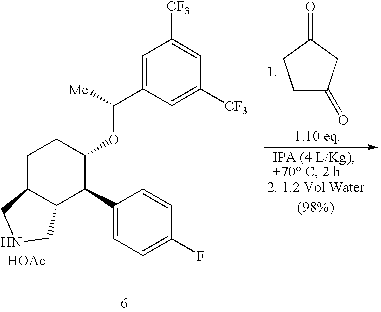

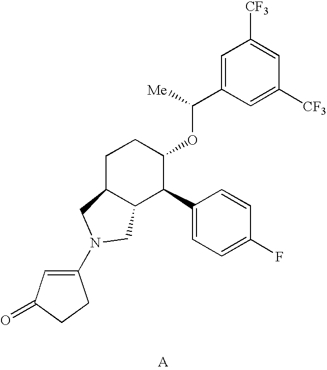

| Materials: | FW | mass | volume | mol | equiv. |
|---|---|---|---|---|---|
| Acetic acid Salt | 535.5 | 7.36 kg | | 13.74 | 1.0 |
| 1,3-cyclopentanedione | 98.10 | 1.48 kg | | 15.12 | 1.1 |
| IPA | | | 30 L | | |
| Water | | | 36 L | | |

A 100 liter flask was charged with IPA (26 L). To this was added the acetic acid salt (7.5 Kg) followed by 1,3-cyclopentanedione (1.51 Kg). The sides of the flask were washed with IPA (4 L) and the mixture is heated to +75° C. for 1 h at which point HPLC indicated that the reaction was complete. To the reaction mixture was then added ⅓ volume of water (10 L) keeping the temperature at +60° C. The batch was seeded (2.00 g, 0.02 wt %) to initiate crystallization. After aging at 50-60° C. for 30 min, the mixture was cooled to 40° C. The remaining water (26 L) was added over a period of 1.25 h and the slurry was aged for 12 hours at rt. The batch was filtered and the wet-cake was washed with 2 bed volumes of 2:1 Water/IPA and then 1 bed volume of water and dried overnight under vacuum/N₂sweep. The resulting wet cake was transferred to a vacuum over and further dried at 45° C. under vacuum with a sweep of nitrogen for 24 h to give 7.45 Kg of API (98% yield).

### EXAMPLE 1

### Making of Form II of Compound A

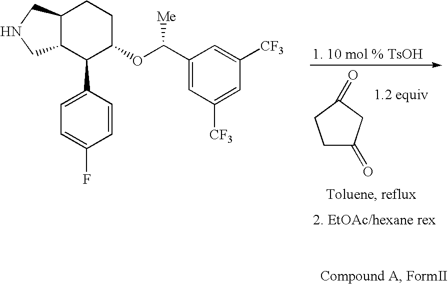

To a solution containing 4.11 g (8.64 mmol) of crude amine starting material in 65 mL of toluene was added 1.02 g (10.04 mmol) of 1,3-cyclopentane dione and 164 mg (0.864 mmol) of p-toluenesulfonic acid hydrate. The resulting mixture was heated to reflux for 3 h, cooled to rt, and concentrated under reduced pressure. To the residue was 100 mL of EtOAc and 100 mL of sat. NaHCO₃, the layers mixed, and allowed to settle. The organic layer was dried over MgSO₄, filtered over a pad of Solka Floc, and the solvent removed under reduced pressure. The resulting solid was re-dissolved in 125 mL of EtOAc and hexane was added to a final volume of 500 mL. The resulting crystalline solid was filtered to give 2.84 g (59%) of Form II of Compound A which was characterized by Physical measurements.

What is claimed is:

1. The crystalline anhydrous Form II of the compound structural formula A

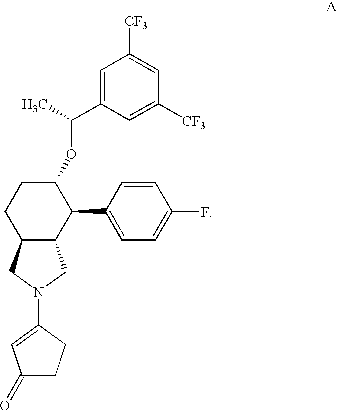

2. The crystalline anhydrous Form II of the compound structural formula A 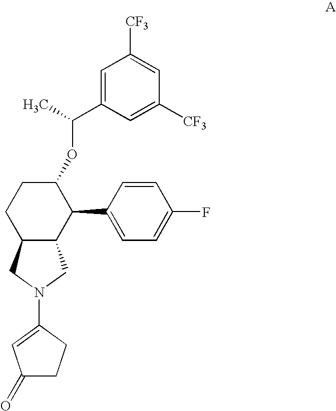
characterized by diffraction peaks obtained from the X-ray powder diffraction pattern corresponding to d-spacings of 7.7, 4.9 and 3.9 angstroms.

3. The crystalline anhydrous Form II of the compound structural formula A according to claim 2 further characterized by diffraction peaks obtained from the X-ray powder diffraction pattern corresponding to d-spacings of 5.3, 4.6 and 3.9 angstroms.

4. The crystalline anhydrous Form II of the compound structural formula A according to claim 2 further characterized by diffraction peaks obtained from the X-ray powder diffraction pattern corresponding to d-spacings of 4.2, 3.8 and 2.8 angstroms.

5. The crystalline anhydrous Form II of the compound structural formula A 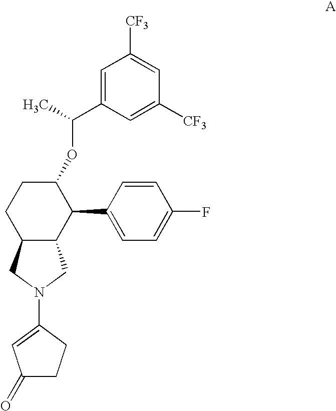
characterized by the X-ray powder diffraction pattern of FIG. 6.

6. The crystalline anhydrous Form II of the compound structural formula A 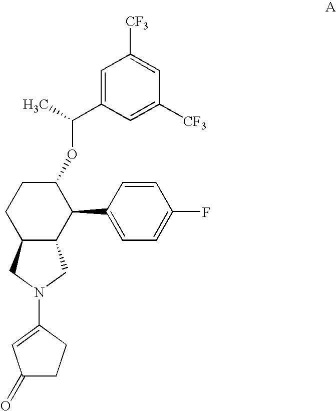
characterized by a solid-state fluorine-19 MAS nuclear magnetic resonance spectrum showing signal at −60.4, −63.4, and −115.3 ppm.

7. The crystalline anhydrous Form II of the compound structural formula A 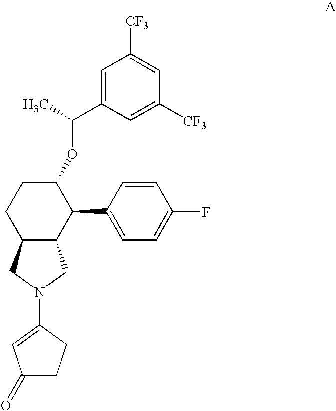
characterized by the solid-state fluorine-19 MAS nuclear magnetic resonance spectrum of FIG. 8.

8. The crystalline anhydrous Form II of the compound structural formula A 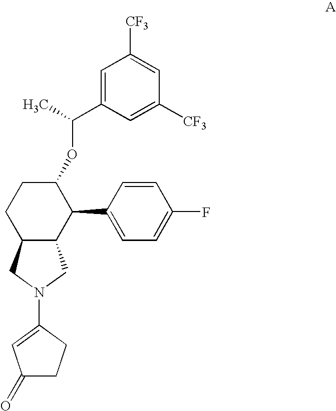
characterized by a melting onset at 218° C.

9. The crystalline anhydrous Form II of the compound structural formula A according to claim 8, further characterized by a peak temperature of 220.3° C.

10. The crystalline anhydrous Form II of the compound structural formula A according to claim 8, further characterized by an enthalpy change of 71.7 J/g.

11. The crystalline anhydrous Form II of the compound structural formula A according to claim 9, further characterized by an enthalpy change of 71.7 J/g. 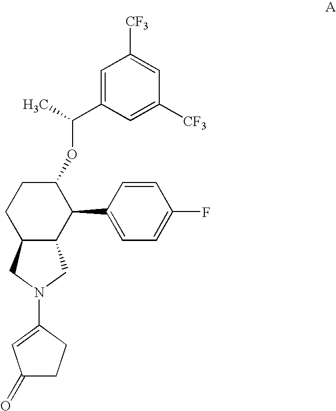
characterized by the DSC curve of FIG. 9.

12. A pharmaceutical composition comprising a compound according to claim 1 and a pharmaceutically acceptable carrier.

13. A method for the manufacture of a medicament for antagonizing the effect of substance P at its receptor site or for the blockade of neurokinin-1 receptors in a mammal comprising combining the compound of claim 1 or a pharmaceutically acceptable salt thereof with a pharmaceutical carrier or diluent.

14. A method for the manufacture of a medicament for the treatment of a physiological disorder associated with an excess of tachykinins in a mammal comprising combining the compound of claim 1 or a pharmaceutically acceptable salt thereof with a pharmaceutical carrier or diluent.

15. A method of antagonizing the effect of substance P at its receptor site or for the blockade of neurokinin-1 receptors in a patient comprising administering said patient an effective amount of a compound according to claim 1.
